(19) United States
(12) Patent Application Publication
(10) Pub. No.: US 2025/0228479 A1
(43) Pub. Date: Jul. 17, 2025

(54) EPIDERMAL BIOSENSOR

(71) Applicant: AGENCY FOR SCIENCE, TECHNOLOGY AND RESEARCH, Singapore (SG)

(72) Inventors: Yuxin LIU, Singapore (SG); Wei Peng GOH, Singapore (SG); Xinting ZHENG, Singapore (SG); Yong YU, Singapore (SG); Chong Li Sherwin TAN, Singapore (SG); Changyun JIANG, Singapore (SG); Ruth Theresia ARWANI, Singapore (SG); Le YANG, Singapore (SG)

(21) Appl. No.: 18/844,874

(22) PCT Filed: Mar. 2, 2023
(86) PCT No.: PCT/SG2023/050125

§ 371 Date: Sep. 6, 2024

(30) Foreign Application Priority Data
Mar. 8, 2022 (SG) 10202202318P

(51) Int. Cl. A61B 5/1477; A61B 5/1486

(52) U.S. Cl. CPC A61B 5/1477; A61B 5/1486; A61B 2562/166

(57) ABSTRACT

An epidermal biosensor comprising a diffusion layer operable to dissolve a solid-phase epidermal analyte, an enzymatic bioreceptor operable to oxidise the dissolved epidermal analyte from the diffusion layer, a transducer having an interface with the diffusion layer, a processor configured to process electrochemical data from the transducer, and a substrate to which the enzymatic bioreceptor and the transducer are attached. The solid-phase epidermal analyte may include water-insoluble cholesterol and water-soluble lactate. The diffusion-solvation layer may comprise agarose hydrogel and additives including glycerol and/or gelatin to improve mechanical robustness and reduce water evaporation rate of the hydrogel, and ethanol and/or Triton X-100 to facilitate solvation and transportation of hydrophobic analytes.

### FIELD OF THE INVENTION

The present invention relates in general to biochemical data monitoring and more particularly to an epidermal biosensor.

### BACKGROUND OF THE INVENTION

Monitoring biochemical data is critical for medical diagnosis, disease monitoring, and health management. Invasiveness and form factor of the sensor are two (2) limiting factors preventing continuous monitoring of important biomarkers. For example, blood tests are invasive, non-continuous, costly and require professional medical settings.

Recent research efforts into non-invasive sensing modalities focused on alternative biofluids such as human sweat. Continuous measurement of biomarker concentration in sweat has been proposed for both fitness tracking and chronic disease monitoring. However, acquisition of sweat from patients and people who are physically inactive is difficult. A typical method of sweat induction involves iontophoresis or administration of drugs such as pilocarpine. However, chronic use of sweat-induction methods causes patient discomfort and other side effects including headache, skin pH changes, lesions, diarrhoea and flu-like symptoms.

Solid-phase biomarkers may be used for diagnosing and monitoring chronic diseases. For example, epidermal solid-phase glucose is positively correlated to diabetes, and potentially more sensitive than urine and capillary blood obtained by fingerstick. Similarly, epidermal solid-phase cholesterol may be used as an important biomarker for hyperlipoproteinemia, coronary artery disease and atherosclerosis. Lactate from insensible perspiration provides valuable health information for cardiovascular diseases. However, current detection methods rely on off-line sample collection from skin and require use of sophisticated instruments such as mass spectrometry (MS) or high-performance liquid chromatography (HPLC), which are difficult to miniaturize for remote patient monitoring and do not provide continuous monitoring.

In view of the foregoing, it would be desirable to provide a low-cost epidermal biosensor that is able to perform continuous solid-phase electrochemical sensing.

### SUMMARY OF THE INVENTION

Accordingly, in a first aspect, the present invention provides an epidermal biosensor. The epidermal biosensor includes a diffusion layer operable to dissolve a solid-phase epidermal analyte, an enzymatic bioreceptor operable to oxidise the dissolved epidermal analyte from the diffusion layer, a transducer having an interface with the diffusion layer, a processor configured to process electrochemical data from the transducer, and a substrate to which the enzymatic bioreceptor and the transducer are attached.

Other aspects and advantages of the invention will become apparent from the following detailed description, taken in conjunction with the accompanying drawings, illustrating by way of example the principles of the invention.

### BRIEF DESCRIPTION OF THE DRAWINGS

Embodiments of the invention will now be described, by way of example only, with reference to the accompanying drawings, in which:

FIG. 1 is an exploded schematic diagram illustrating an epidermal biosensor in accordance with an embodiment of the present invention;

FIG. 2 is a cross-sectional schematic diagram illustrating operation of the epidermal biosensor of FIG. 1;

FIG. 3 is a photograph of an epidermal biosensor in accordance with an embodiment of the present invention attached to human skin;

FIG. 4 is a schematic diagram illustrating an application of the epidermal biosensor of FIG. 1;

FIG. 5A is a graph illustrating sensor sensitivity and working time at various glycerol/water ratios for characterization of a solid-phase lactate interface;

FIG. 5B is a graph illustrating amperometric measurements taken over time using lactate oxidase-based sensors;

FIG. 5C is a graph illustrating a calibration curve of current density versus area density of a solid-phase lactate;

FIG. 5D is a graph illustrating selectivity characterization of the solid-phase lactate interface using chronoamperometry;

FIG. 6A is a graph illustrating sensor sensitivity and working time at various gelatin ratios for characterization of a solid-phase cholesterol interface;

FIG. 6B is a graph illustrating amperometric measurements taken over time using cholesterol oxidase-based sensors;

FIG. 6C is a graph illustrating a calibration curve of current density versus area density of a solid-phase cholesterol;

FIG. 6D is a graph illustrating selectivity characterization of the solid-phase cholesterol interface using chronoamperometry;

FIGS. 7A and 7B are graphs providing a comparison of lactate predicted by a solid-phase electrochemical sensor and lactate measured by a commercial colorimetric assay kit; and

FIGS. 7C and 7D are graphs providing a comparison of cholesterol predicted by a solid-phase electrochemical sensor and cholesterol measured by a commercial colorimetric assay kit.

### DETAILED DESCRIPTION OF EXEMPLARY EMBODIMENTS

The detailed description set forth below in connection with the appended drawings is intended as a description of presently preferred embodiments of the invention, and is not intended to represent the only forms in which the present invention may be practiced. It is to be understood that the same or equivalent functions may be accomplished by different embodiments that are intended to be encompassed within the scope of the invention.

The term “diffusion layer” as used herein refers to a thickness of a porous material that permits movement of a substance through the porous material based on concentration differences.

The term “enzymatic bioreceptor” as used herein refers to a biological substance that is able to catalyse a reaction with selectivity.

The term “hydrogel material” as used herein refers to a three-dimensional (3D) network of hydrophilic polymers that swells in water and holds a large amount of water, while maintaining its structure due to chemical or physical cross-linking of individual polymer chains.

The term “about” as used herein refers to both numbers in a range of numerals and is also used to indicate that a value includes the standard deviation of error for the device or method being employed to determine the value. The term “about” as used herein can allow for a degree of variability in a value or range, for example, within 10%, within 5%, or within 1% of a stated value or of a stated limit of a range.

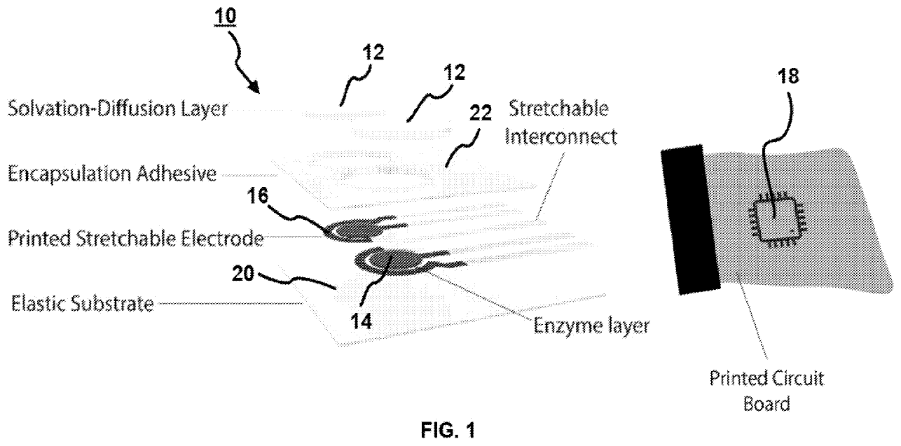

Referring now to FIG. 1, an epidermal biosensor 10 is shown. The epidermal biosensor 10 includes a diffusion layer 12 operable to dissolve a solid-phase epidermal analyte, an enzymatic bioreceptor 14 operable to oxidise the dissolved epidermal analyte from the diffusion layer 12, a transducer 16 having an interface with the diffusion layer 12, a processor 18 configured to process electrochemical data from the transducer 16, and a substrate 20 to which the enzymatic bioreceptor 14 and the transducer 16 are attached. In the embodiment shown, an adhesive layer 22 encapsulates a portion of the substrate 20. A plurality of interconnects 24 may be provided to electronically connect the transducer 16 to the processor 18.

Various elements of the epidermal biosensor 10 may be made of flexible and/or stretchable or elastic materials. The epidermal biosensor 10 provides a stretchable biochemical interface for epidermal solid-phase biomarkers. Fully integrated and wearable, the stretchable electrochemical sensor 10 is capable of continuous detection of biochemicals in the solid-phase. The epidermal biosensor 10 may further be integrated with a battery and a mobile application.

In use, the diffusion layer 12 is in direct contact with human skin and solvates solid-phase biomolecules present on the human skin (epidermal biomolecules). A solvation-diffusion process of solid-phase analytes occurs in the diffusion layer 12—the solvation-diffusion layer 12 dissolves solid analytes and allows diffusion of the dissolved analytes to the enzymatic bioreceptor 14 and the transducer 16.

The diffusion layer 12 may include a matrix or three-dimensional network of hydrophilic polymers such as, for example, a hydrogel material. To allow electrochemical reactions to occur, the hydrogel material was designed and engineered to solvate solid-phase molecules, allowing subsequent diffusion. More particularly, the hydrogel material serves dual functions of solvating the solid-phase analyte and facilitating transport from the skin to a sensing interface; solid analytes solvate and diffuse in the hydrogel material. A phase transition from solid to liquid takes place in the hydrogel material. The hydrogel material may be formulated and designed for solvation process from solid to liquid and diffusion process for a targeted solid-phase analyte. The solvation-diffusion layer 12 made of hydrogel materials allows for solid-to-liquid transformation of the solid analytes within a hydrogel matrix, enabling electrochemical quantification of areal density of solid-phase biomolecules present on human skin (epidermal biomolecules). Advantageously, due to the use of hydrogel material, the epidermal biosensor 10 is less susceptible to motion artifacts. The electrochemical interface provided by the epidermal biosensor 10 is also more stable with hydrogel than a liquid interface (sweat) on dynamically stretching skin. In one embodiment, the hydrogel material 12 may consist of between about 0.1 percent weight per volume (% w/v) and about 4% w/v agarose hydrogel. If the ratio of agarose in the hydrogel is too high, the diffusion of ions and analytes may be impeded, giving poor sensing results.

Working time of the diffusion-solvation layer 12 may be extended by introducing a high-boiling point biocompatible additive such as, for example, glycerol. Glycerol may be added into the hydrogel to reduce or slow down water evaporation rate and achieve a long working time, while not severely reducing sensor performance. To improve mechanical robustness of the hydrogel and reduce the water evaporation rate of the hydrogel, different weight percentages of gelatin may be added. Accordingly, to provide higher working time, the diffusion layer 12 may further include glycerol and/or gelatin. In one embodiment, the hydrogel material 12 may consist of between about 0.1% w/v and about 10% w/v gelatin hydrogel.

For water-insoluble biomarkers, additives such as, for example, ethanol and/or 2-[4-(2,4,4-trimethylpentan-2-yl) phenoxy]ethanol (Triton X-100) may be included to increase solubility in the diffusion layer 12 in order to facilitate solvation and transportation of hydrophobic analytes. Accordingly, the diffusion layer 12 may further include a surfactant. The surfactant may be added into the hydrogel for hydrophobic species. The surfactant may include between about 0.1% w/v and about 10% w/v 2-[4-(2,4,4-trimethylpentan-2-yl) phenoxy]ethanol (Triton X-100). The diffusion layer 12 may further include between about 0.1% w/v and about 10% w/v ethanol. For example, hydrogel may be modified with certain amount of ethanol (2%) and surfactant (2% Triton-X 100) for water insoluble biomarkers.

If the diffusion layer 12 is too thick, diffusion may take too long. However, if the diffusion layer 12 is too thin, water tends to evaporate from the diffusion layer 12 more quickly at body temperature. To optimize the epidermal biosensor 10 to allow fast response and prolonged working time at the same time, the diffusion layer 12 may have a thickness of between about 300 microns (μm) and about 1.5 millimetres (mm).

Through engineering of the solvation-diffusion layer 12, the epidermal biosensor 10 may be used to detect both water-soluble analytes (for example, lactate) and water-insoluble analytes (for example, cholesterol) for monitoring of chronic conditions such as, for example, cardiovascular diseases.

The enzymatic bioreceptor 14 may be provided as an enzyme functionalization layer where natural enzymes oxidize the analytes and generate hydrogen peroxide. The enzymatic layer or enzymatic bioreceptor 14 provides a layer of enzymes that converts target biomolecules into other molecules that allow measurement of electron transfer.

The transducer 16 may include a screen-printed electrode. Electrochemical reactions occur at the hydrogel-electrode interface. To provide a screen-printed stretchable electrode, the electrode may include graphite and poly(3,4-ethylenedioxythiophene) polystyrene sulfonate (PEDOT:PSS). The electrode may further include at least one selected from a group consisting of iron (II, III) hexacyanoferrate (II, III) (Prussian blue (PB)), waterborne polyurethane, dimethyl sulfoxide (DMSO) and (3-glycidyloxypropyl) trimethoxy silane (GPTMS). Advantageously, Prussian blue (PB) may be used as a redox mediator to reduce overpotential required for detection of hydrogen peroxide. Advantageously, inclusion of viscoelastic WPU as a secondary polymer network helps improve stability and sensitivity of the electrode and also helps improve stretchability of the epidermal biosensor 10. Introduction of the stretchable WPU enables the printed electrode ink/paste to be stretchable, allowing the stretchable version of the electrode to be able to sustain 30% strain without performance degradation. DMSO helps to improve conductivity of the electrode material, while GPTMS helps to improve stability of the electrode material in an aqueous environment.

The processor 18 may be integrated with a printed circuit board (PCB) or flexible printed circuit board (fPCB). A front-end circuit on the PCB may collect and process the electrochemical data.

The substrate 20 is elastic in nature and may be made of a polymer such as, for example, styrene-ethylene-butylene-styrene (SEBS).

The interconnects 24 may be silver-based conductive traces formed by screen-printing and may be made of a polymeric material for stretchability, allowing the interconnects 24 to sustain substantial strain without breaking.

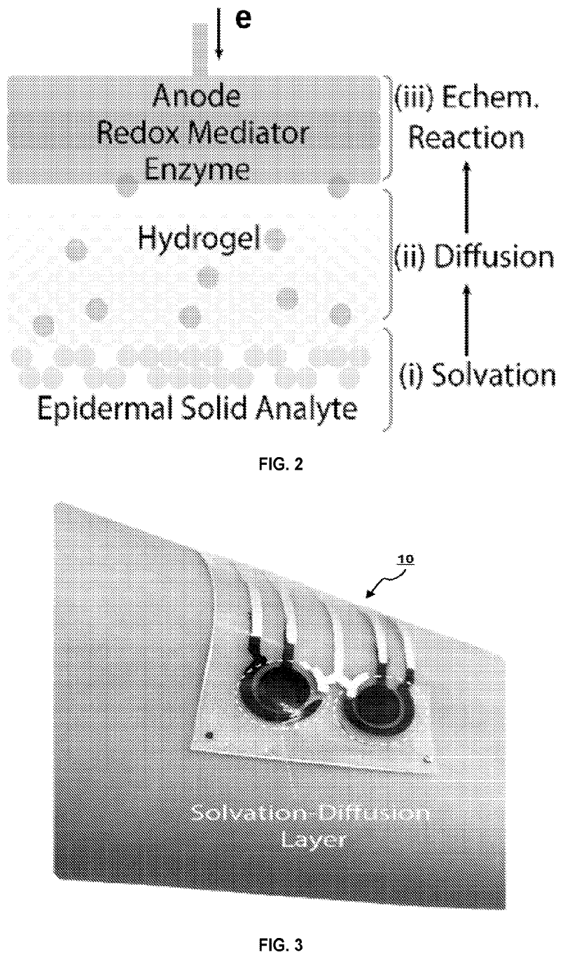

Referring now to FIG. 2, a cross-section of a working electrode is shown illustrating a three (3)-step process for epidermal solid analytes, in particular, solvation, diffusion and electrochemical reaction. As can be seen from FIG. 2, electrochemical reaction occurs at the hydrogel-electrode interface.

Referring now to FIG. 3, the epidermal biosensor 10 is shown placed on human skin and conforming to a curvilinear surface of a forearm of a user. Being stretchable, the epidermal biosensor 10 is able to accommodate body movements without causing user discomfort and severe motion artifacts. As shown in FIG. 3, the solvation-diffusion layer, encircled by dashed circles, is directly in contact with stratum corneum to allow transport of solid-phase analytes to an electrode surface.

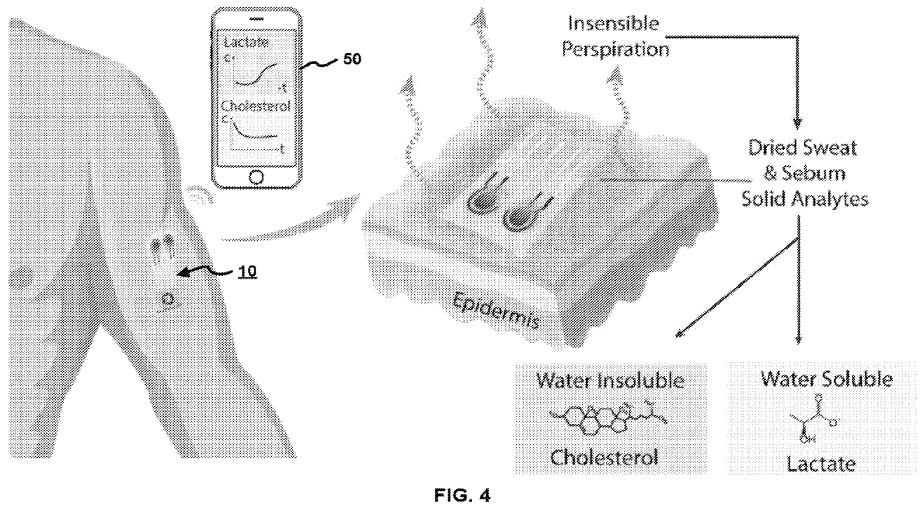

Referring now to FIG. 4, a use case of the epidermal biosensor 10 providing a stretchable biochemical interface for solid epidermal analytes is shown. As can be seen from FIG. 4, the epidermal biosensor 10 may be placed on skin for detection of epidermal solid-phase biomarkers including water-insoluble cholesterol and water-soluble lactate. The epidermal biosensor 10 may be wirelessly connected to a smartphone 50 for reading out areal density of the solid-phase biomarkers. Electrochemical data from the epidermal biosensor 10 may be sent to the smartphone 50 via Bluetooth technology. The data may then be displayed to the user, a family member and a caregiver for tracking chronic diseases. Advantageously, the epidermal biosensor 10 enables in situ detection of solid-phase biomarkers including dried sweat and secretion of sebum. Further advantageously, the stretchable solid-phase sensor 10 eliminates the need for sweat-induction through drugs or exercise and offers a comfortable and non-invasive interface for reliable acquisition of applied health signals outside of a hospital or clinical environment.

### EXAMPLES

### Example 1

Sensing performance of a solid-phase lactate interface of an electrochemical sensor was experimentally characterized. A thin layer of hydrogel made of 2 wt % low-melting agarose was selected to constitute a solvation-diffusion layer because of its fast diffusion response, low cost, and enzyme compatibility. Glycerol was added into the hydrogel to reduce the water evaporation rate and achieve a long working time.

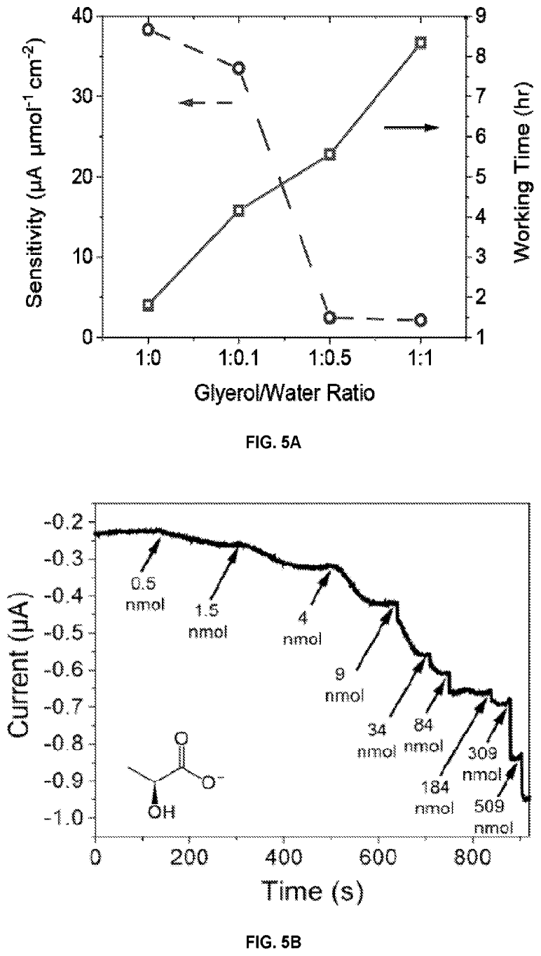

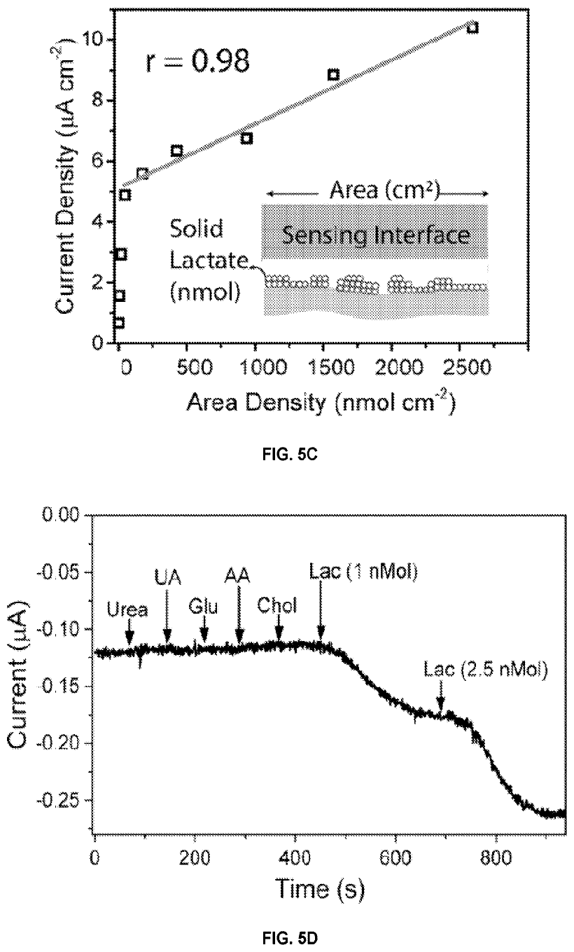

Referring now to FIG. 5A, optimization of sensor sensitivity and working time (limited by water evaporation) at various glycerol/water ratios for characterization of the solid-phase lactate interface is shown. As can be seen from FIG. 5A, the working time is increased from 1.8 hr in purely water-based hydrogel (no glycerol) to 4.2 hr for hydrogel with a glycerol/water ratio of 0.1:1. However, glycerol adversely affected the sensitivity, possibly caused by the impediment of the diffusion of lactate towards the hydrogel-electrode interface. As a compromise, the sensitivity of the sensor is reduced from 32.5 μA μmol⁻¹ cm⁻² in the absence of glycerol to 10.3 μA μmol⁻¹ cm⁻² for a glycerol/water ratio of 0.1:1. Excessive glycerol further hampered sensor sensitivity and an optimal glycerol/water ratio of 0.1:1 was used throughout subsequent experiments.

Referring now to FIG. 5B, amperometric measurements using lactate oxidase-based sensors are shown. Prussian blue was used as a redox mediator.

Referring now to FIG. 5C, a calibration curve of current density versus area density of solid-phase lactate is shown. As indicated in FIG. 5C, Pearson correlation coefficient r is 0.98. A sensing interface at the solvation-diffusion layer and solid-phase lactate is illustrated in an inset of FIG. 5C. The area density is calculated by the solid lactate divided by the contact area at the sensing interface.

FIGS. 5B and 5C show the amperometric detection of the solid-phase lactate. Excellent linearity between 45.9 nmol/cm² to 2593.6 nmol/cm² lactate was observed with Pearson's correlation coefficient of 0.98. The limit of detection (LOD) is 2.5 nmol/cm².

Referring now to FIG. 5D, selectivity characterization using chronoamperometry, where interference molecules (i.e. urea, uric acid (UA), glucose, ascorbic acid (AA), cholesterol) in solid-phase were applied on the solid-phase lactate sensor, is shown. The solid-phase lactate sensor exhibited excellent selectivity over other solid epidermal analytes such as urea, uric acid (UA), glucose, ascorbic acid (AA), and cholesterol in physiological relevant amounts.

### Example 2

For water-insoluble biomarkers, additives such as ethanol and Triton X-100 were used to increase solubility in the hydrogel. For a solid-phase cholesterol sensor, 2 v/v % ethanol and 2 v/v % Triton X-100 were dissolved into a low-melting agarose solution. To further improve mechanical robustness of the hydrogel and reduce water evaporation rate of the hydrogel, different weight percentages of gelatin were added.

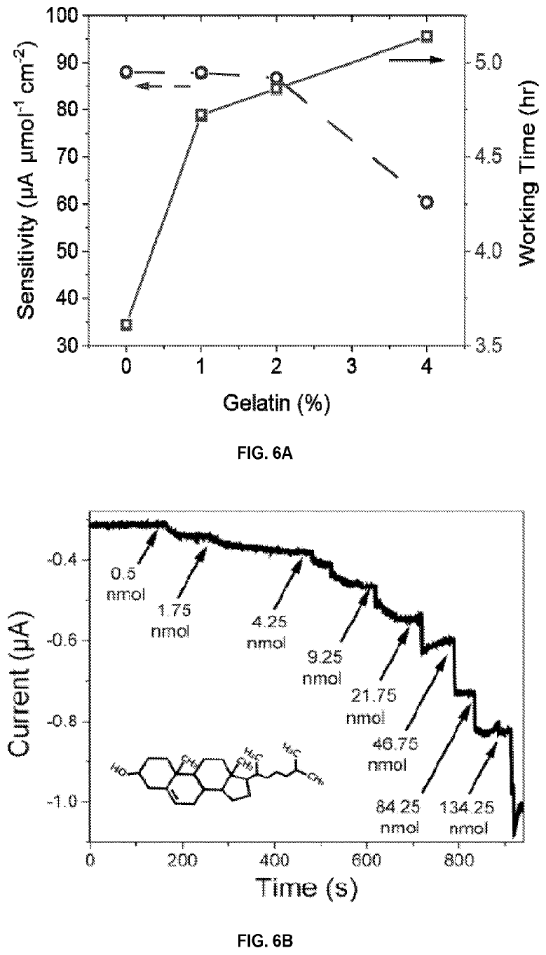

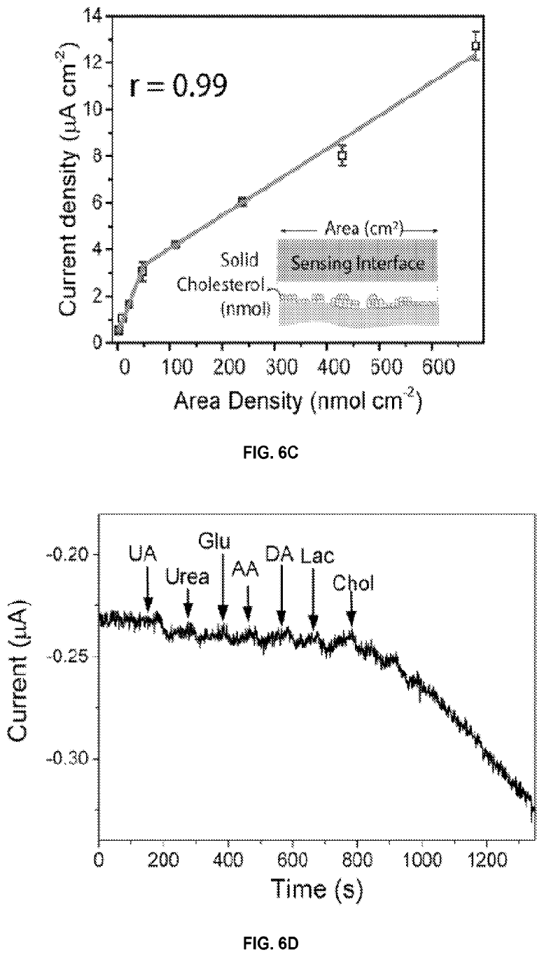

Referring now to FIG. 6A, optimization of sensor sensitivity and working time (limited by water evaporation) at various gelatin ratios for characterization of the solid-phase cholesterol interface is shown. As can be seen from FIG. 6A, the sensitivity of the cholesterol sensor was stable from 0% to 2% and decreased substantially at 4%. Consequently, 2% gelatin was used to achieve a relatively long working time (4.8 hr of continuous amperometry detection) and good sensitivity in the following experiments.

Referring now to FIG. 6B, amperometric measurements using cholesterol oxidase-based sensors are shown. Prussian blue was used as a redox mediator.

Referring now to FIG. 6C, a calibration curve of current density versus area density of solid-phase cholesterol is shown. As indicated in FIG. 6C, the Pearson's correlation coefficient r is 0.99 for both linear regions. A sensing interface at the solvation-diffusion layer and solid-phase cholesterol is illustrated in an inset of FIG. 6C. The area density is calculated by the solid cholesterol divided by the contact area at the sensing interface. Two linear regions were observed between 2.5 nmol/cm² and 47.1 nmol/cm² and between 47.1 nmol/cm² and 684.1 nmol/cm² with LOD of 2.5 nmol/cm².

Referring now to FIG. 6D, selectivity characterization using chronoamperometry, where interference molecules (i.e. urea, UA, glucose, dopamine (DA), AA, lactate) were tested on the solid-phase cholesterol sensor, is shown. The device showed excellent selectivity against other solid-phase epidermal analytes, such as urea, uric acid, glucose, ascorbic acid dopamine, and lactate.

The response of the solid-phase lactate and cholesterol may be recorded via a printed circuit board (PCB) and wirelessly transmitted to a smartphone via Bluetooth. Although only cholesterol and lactate were demonstrated, other epidermal biomarkers such as glucose may be detected using the same device structure and sensing mechanism.

### Example 3

Next, the solid-phase electrochemical sensors were tested using ex vivo human samples. The sample was prepared by drop casting 5 μl of human sweat and allowing it to dry at ambient temperature. For ex vivo validation of the solid-phase electrochemical sensors, sensor accuracy was quantified by comparing solid-phase electrochemical sensors with commercial assay kits.

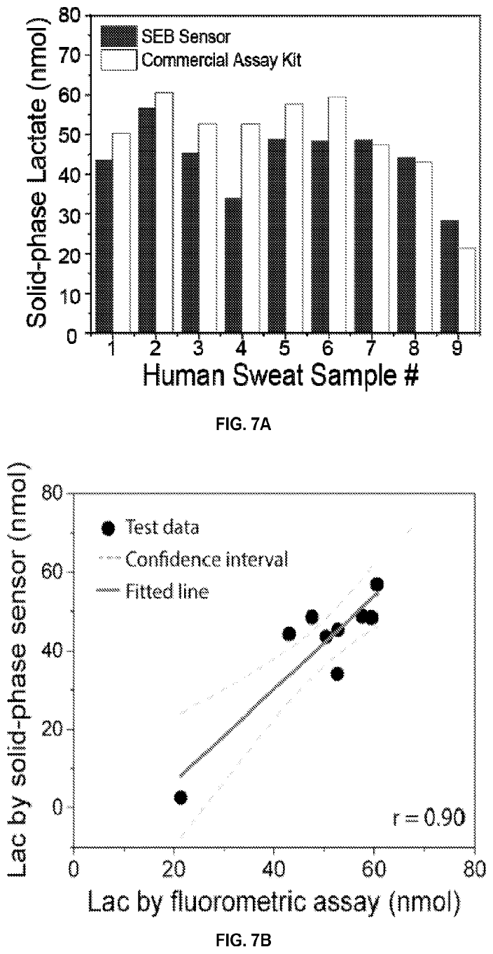

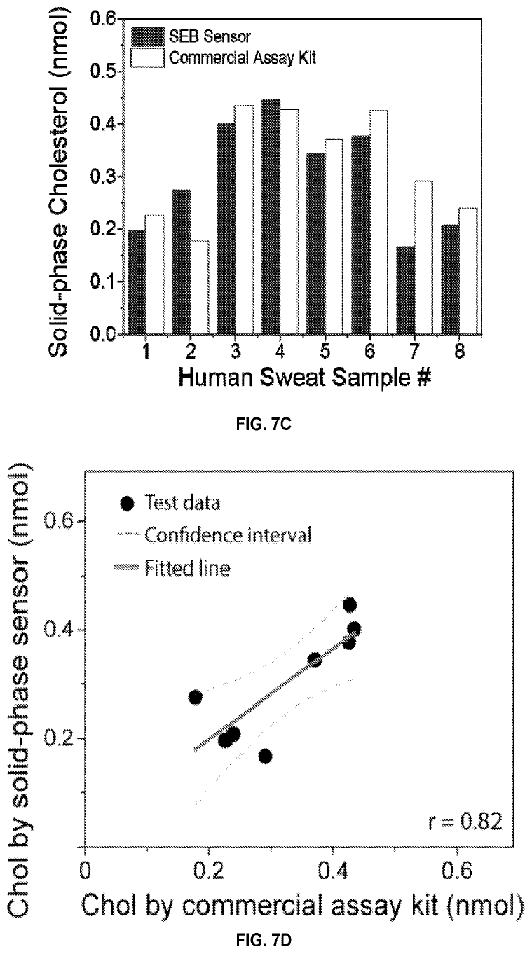

Referring now to FIGS. 7A and 7B, a comparison of lactate predicted by a solid-phase electrochemical sensor and lactate measured by a commercial colorimetric assay kit is provided. The Pearson's correlation coefficient r is 0.90. The dashed line in FIG. 7B denotes confidence interval (95%).

Referring now to FIGS. 7C and 7D, a comparison of the cholesterol predicted by a solid-phase electrochemical sensor and cholesterol measured by a commercial colorimetric assay kit. The Pearson's correlation coefficient r is 0.82. The dashed line in FIG. 7D denotes confidence interval (95%).

A positive relationship between biomarker amount by commercial kit and that by the solid-phase sensors was observed for both lactate as seen from FIGS. 7A and 7B and cholesterol as seen from FIGS. 7C and 7D with Pearson's correlation coefficient of 0.90 and 0.82, respectively. The ex vivo testing demonstrated good reliability of the sensor for quantification of human biological samples in solid-phase.

As is evident from the foregoing discussion, the present invention provides an epidermal biosensor that allows for continuous detection of epidermal solid analytes on human skin. Apart from measuring liquid analytes in a specific biofluid such as sweat, urine, interstitial fluids, the epidermal biosensor of the present invention enables measurement of solid analytes resulting from dried sweat or insensible perspiration, which has clinical relevance in predicting and monitoring chronic diseases. The present invention provides a sensing modality to measure solid biomarkers that are present on the surface of human skin, therefore eliminating the need for sweat induction or physical exercise. Advantageously, with the present invention, applied actionable health signals can be readily measured by simply placing a stretchable wearable patch on skin, allowing remote health monitoring, preventative medicine, and telemedicine. The epidermal biosensor of the present invention is non-invasive, low cost and may be wearable at home. By eliminating the need for exercise, compliance may be improved. Advantageously, the epidermal biosensor of the present invention is able to provide real-time, in situ and continuous detection of solid epidermal analytes without invasiveness (e.g., blood collection), or additional medical procedures (e.g., sweat induction and urine collection). The epidermal biosensor of the present invention also provides a generic platform to detect both hydrophilic and hydrophobic biomarkers. Further advantageously, the skin-integrated, stretchable wearable biochemical sensor of the present invention does not require complicated equipment such as mass spectrometry or/and liquid chromatography.

While preferred embodiments of the invention have been described, it will be clear that the invention is not limited to the described embodiments only. Numerous modifications, changes, variations, substitutions and equivalents will be apparent to those skilled in the art without departing from the scope of the invention as described in the claims.

Further, unless the context clearly requires otherwise, throughout the description and the claims, the words “comprise”, “comprising” and the like are to be construed in an inclusive as opposed to an exclusive or exhaustive sense; that is to say, in the sense of “including, but not limited to”.

1. An epidermal biosensor, comprising:
a diffusion layer operable to dissolve a solid-phase epidermal analyte;
an enzymatic bioreceptor operable to oxidise the dissolved epidermal analyte from the diffusion layer;
a transducer having an interface with the diffusion layer;
a processor configured to process electrochemical data from the transducer; and
a substrate to which the enzymatic bioreceptor and the transducer are attached.

2. The epidermal biosensor of claim 1, wherein the diffusion layer comprises a matrix of hydrophilic polymers.

3. The epidermal biosensor of claim 1, wherein the diffusion layer comprises a hydrogel material.

4. The epidermal biosensor of claim 3, wherein the hydrogel material consists of between about 0.1 percent weight per volume (% w/v) and about 4% w/v agarose hydrogel.

5. The epidermal biosensor of claim 1, wherein the diffusion layer further comprises at least one selected from a group consisting of glycerol and gelatin.

6. The epidermal biosensor of claim 3, wherein the hydrogel material consists of between about 0.1% w/v and about 10% w/v gelatin hydrogel.

7. The epidermal biosensor of claim 1, wherein the diffusion layer further comprises a surfactant.

8. The epidermal biosensor of claim 7, wherein the surfactant comprises between about 0.1% w/v and about 10% w/v 2-[4-(2,4,4-trimethylpentan-2-yl) phenoxy]ethanol.

9. The epidermal biosensor of claim 1, wherein the diffusion layer further comprises between about 0.1% w/v and about 10% w/v ethanol.

10. The epidermal biosensor of claim 1, wherein the diffusion layer has a thickness of between about 300 microns (μm) and about 1.5 millimetres (mm).

11. The epidermal biosensor of claim 1, wherein the transducer comprises a screen-printed electrode.

12. The epidermal biosensor of claim 11, wherein the electrode comprises graphite and poly(3,4-ethylenedioxythiophene) polystyrene sulfonate (PEDOT:PSS).

13. The epidermal biosensor of claim 12, wherein the electrode further comprises at least one selected from a group consisting of iron (II, III) hexacyanoferrate (II, III), waterborne polyurethane, dimethyl sulfoxide (DMSO) and (3-glycidyloxypropyl) trimethoxy silane (GPTMS).

14. The epidermal biosensor of claim 1, further comprising a plurality of interconnects electronically connecting the transducer to the processor.

15. The epidermal biosensor of claim 1, wherein the substrate comprises styrene-ethylene-butylene-styrene (SEBS).

16. The epidermal biosensor of claim 1, further comprising an adhesive layer encapsulating a portion of the substrate.
